(19) United States
(12) Patent Application Publication
(10) Pub. No.: US 2013/0177554 A1
(43) Pub. Date: Jul. 11, 2013

(54) NEUROPILIN AS A BIOMARKER FOR BEVACIZUMAB COMBINATION THERAPIES

(71) Applicant: Hoffmann-La Roche Inc., Nutley, NJ (US)

(72) Inventors: Paul DELMAR, Basel (CH); Dorothee Foernzler, Lenzburg (CH); Stefan Scherer, South San Francisco, CA (US)

(73) Assignee: Hoffmann-La Roche Inc., Nutley, NJ (US)

(21) Appl. No.: 13/802,205

(22) Filed: Mar. 13, 2013

Related U.S. Application Data
(63) Continuation of application No. 13/737,586, filed on Jan. 9, 2013.
Application No. 13/737,586 is a continuation of application No. PCT/EP2011/063932, filed on Aug. 12, 2011.

(30) Foreign Application Priority Data
Aug. 13, 2010 (EP) 10172812.9

(51) Int. Cl. G01N 33/68; A61K 45/00; A61K 39/395

(52) U.S. Cl. CPC G01N 33/6893; A61K 39/39558; A61K 45/00
USPC 424/133.1; 435/7.23

(57) ABSTRACT

The present invention provides methods for improving treatment effect in a patient suffering from gastric cancer, in particular, adenocarcinoma of the stomach or gastro-esophageal junction (“GEJ”), by treatment with bevacizumab (Avastin®) in combination with a chemotherapy regimen by determining the expression level of neuropilin relative to a control level determined in patients suffering from gastric cancer, in particular, adenocarcinoma of the stomach or gastro-esophageal junction (“GEJ”). The improved treatment effect may be improved overall survival or improved progression free survival. The present invention further provides for methods for assessing the sensitivity or responsiveness of a patient to bevacizumab (Avastin®) in combination with a chemotherapy regimen, by determining the expression level of neuropilin relative to a control level determined in patients suffering from gastric cancer, in particular, adenocarcinoma of the stomach or gastro-esophageal junction (“GEJ”).

### CROSS-REFERENCE TO RELATED APPLICATIONS

This application is a continuation of U.S. application Ser. No. 13/737,586, filed Jan. 9, 2013, which is a continuation of international application PCT/EP2011/063932, filed Aug. 12, 2011 which claims priority from European Patent Application 10172812.9, filed Aug. 13, 2010, the contents of which are incorporated herein by reference.

The present invention provides methods for improving treatment effect in a patient suffering from gastric cancer, in particular, adenocarcinoma of the stomach or gastro-esophageal junction (“GEJ”), by treatment with bevacizumab (Avastin®) in combination with a chemotherapy regimen by determining the expression level of neuropilin relative to a control level determined in patients suffering from gastric cancer, in particular, adenocarcinoma of the stomach or gastro-esophageal junction (“GEJ”). The improved treatment effect may be improved overall survival or improved progression free survival. The present invention further provides for methods for assessing the sensitivity or responsiveness of a patient to bevacizumab (Avastin®) in combination with a chemotherapy regimen, by determining the expression level of neuropilin relative to a control level determined in patients suffering from gastric cancer, in particular, adenocarcinoma of the stomach or gastro-esophageal junction (“GEJ”).

Accordingly, the present invention relates to the identification and selection of one or more biomarkers of gastric cancer, in particular, adenocarcinoma of the stomach or gastro-esophageal junction (“GEJ”), that correlate with sensitivity or responsiveness to angiogenesis inhibitors, e.g., bevacizumab (Avastin®), in combination with chemotherapeutic regimens, such as capecitabine- or 5-fluorouracil-based chemotherapies. In certain aspects, the invention relates to the use of the tumor specific expression of neuropilin determined relative to controls established in patients suffering from gastric cancer, in particular, adenocarcinoma of the stomach or GEJ, to identify patients sensitive or responsive to the addition of angiogenesis inhibitors, e.g., bevacizumab (Avastin®), to standard chemotherapies. The invention also relates to methods for improving treatment effect in a patient suffering from gastric cancer, in particular, adenocarcinoma of the stomach or GEJ, by the addition of angiogenesis inhibitors, e.g., bevacizumab (Avastin®), to standard chemotherapies, e.g., capecitabine- or 5-fluorouracil-based chemotherapies, by determining the tumor specific expression level of neuropilin relative to a control established in patients suffering from gastric cancer, in particular, adenocarcinoma of the stomach or GEJ. Treatment effect includes the clinical parameters overall survival and progression free survival. The invention also provides for kits and compositions for identification of patients sensitive or responsive to angiogenesis inhibitors, in particular, bevacizumab (Avastin®), which patients are determined and defined in accordance with the methods described herein.

Angiogenesis is necessary for cancer development, regulating not only primary tumor size and growth but also impacting invasive and metastatic potential. Accordingly, the mechanisms mediating angiogenic processes have been investigated as potential targets for directed anti-cancer therapies. Early in the study of angiogenic modulators, the vascular endothelial growth factor (VEGF) signalling pathway was discovered to preferentially regulate angiogenic activity in multiple cancer types and multiple therapeutics have been developed to modulate this pathway at various points. These therapies include, among others, bevacizumab, sunitinib, sorafenib and vatalanib. Although the use of angiogenic inhibitors in the clinic has shown success, not all patients respond or fail to fully respond to angiogenesis inhibitor therapy. The mechanism(s) underlying such incomplete response is unknown. Therefore, there is an increasing need for the identification of patient subgroups sensitive or responsive to anti-angiogenic cancer therapy.

While a number of angiogenesis inhibitors are known, the most prominent angiogenesis inhibitor is Bevacizumab (Avastin®). Bevacizumab is a recombinant humanized monoclonal IgG1 antibody that specifically binds and blocks the biological effects of VEGF (vascular endothelial growth factor). VEGF is a key driver of tumor angiogenesis—an essential process required for tumor growth and metastasis, i.e., the dissemination of the tumor to other parts of the body. Avastin® is approved in Europe for the treatment of the advanced stages of four common types of cancer: colorectal cancer, breast cancer, non-small cell lung cancer (NSCLC) and kidney cancer, which collectively cause over 2.5 million deaths each year. Over half a million patients have been treated with Avastin® so far, and a comprehensive clinical program with over 450 clinical trials is investigating the further use of Avastin in the treatment of multiple cancer types (including colorectal, breast, non-small cell lung, brain, gastric, ovarian and prostate) in different settings (e.g., advanced or early stage disease). Importantly, Avastin® has shown promise as a co-therapeutic, demonstrating efficacy when combined with a broad range of chemotherapies and other anti-cancer treatments. Phase-III studies have been published demonstrating the beneficial effects of combining bevacizumab with standard chemotherapeutic regimens (see, e.g., Kang et al., 2010, J. Clin. Oncol., 28:18s (suppl. abstr. LBA4007); Saltz et al., 2008, J. Clin. Oncol., 26:2013-2019; Yang et al., 2008, Clin. Cancer Res., 14:5893-5899; Hurwitz et al., 2004, N Engl. J. Med., 350:2335-2342). However, as in previous studies of angiogenic inhibitors, some of these phase-III studies have shown that a portion of patients experience incomplete response to the addition of bevacizumab (Avastin®) to their chemotherapeutic regimens.

Accordingly, there is a need for methods of determining those patients that respond or are likely to respond to combination therapies comprising angiogenesis inhibitors, in particular, bevacizumab (Avastin®). Thus, the technical problem underlying the present invention is the provision of methods and means for the identification of (a) patient(s) suffering from or prone to suffer from gastric cancer, in particular, adenocarcinoma of the stomach or GEJ, who may benefit from the addition of angiogenesis inhibitors, in particular, bevacizumab (Avastin®), to chemotherapeutic regimens, e.g., capecitabine- or 5-fluorouracil-based chemotherapies.

The technical problem is solved by provision of the embodiments characterized in the claims.

The present invention, therefore, provides a method for improving treatment effect in a patient suffering from gastric cancer, in particular, adenocarcinoma of the stomach or GEJ, by adding bevacizumab to a chemotherapy regimen, said method comprising:
(a) determining the expression level of neuropilin in a patient sample; and
(b) administering bevacizumab in combination with a chemotherapy regimen to the patient having a decreased level of neuropilin relative to a control level determined in patients suffering from gastric cancer, in particular, adenocarcinoma of the stomach or GEJ.
   The improved treatment effect may be the clinical parameter overall survival or may be progression free survival.

In other embodiments, the present invention relates to an in vitro method for the identification of a patient responsive to or sensitive to the addition of bevacizumab to a chemotherapy regimen, said method comprising determining the expression level of neuropilin in a sample from a patient suspected to suffer from or being prone to suffer from gastric cancer, in particular, adenocarcinoma of the stomach or GEJ, whereby decreased level of neuropilin relative to a control level determined in patients suffering from gastric cancer, in particular, adenocarcinoma of the stomach or GEJ, is indicative of a sensitivity of the patient to the addition of bevacizumab to said regimen.

Accordingly, the present invention solves the identified technical problem in that it was surprisingly shown that the tumor specific expression level of neuropilin in a given patient, relative to a control level determined in patients diagnosed with gastric cancer, in particular, adenocarcinoma of the stomach or GEJ, correlates with treatment effect in those patients administered an angiogenesis inhibitor in combination with a chemotherapy regimen. Variation in the tumor specific expression level of neuropilin was surprisingly identified as a marker/predictor for the improved progression-free survival and/or improved overall survival of gastric cancer patients in response to the addition of bevacizumab (Avastin®) to capecitabine- or 5-fluorouracil-based chemotherapeutic regimens. Specifically, gastric cancer patients exhibiting a response or sensitivity to the addition of bevacizumab (Avastin®) to chemotherapy regimens were identified to have decreased expression of neuropilin relative to a control level established in samples obtained from patients suffering from or diagnosed with gastric cancer, in particular, adenocarcinoma of the stomach or GEJ. The terms “marker” and “predictor” can be used interchangeably and refer to the expression level of neuropilin as described and defined herein.

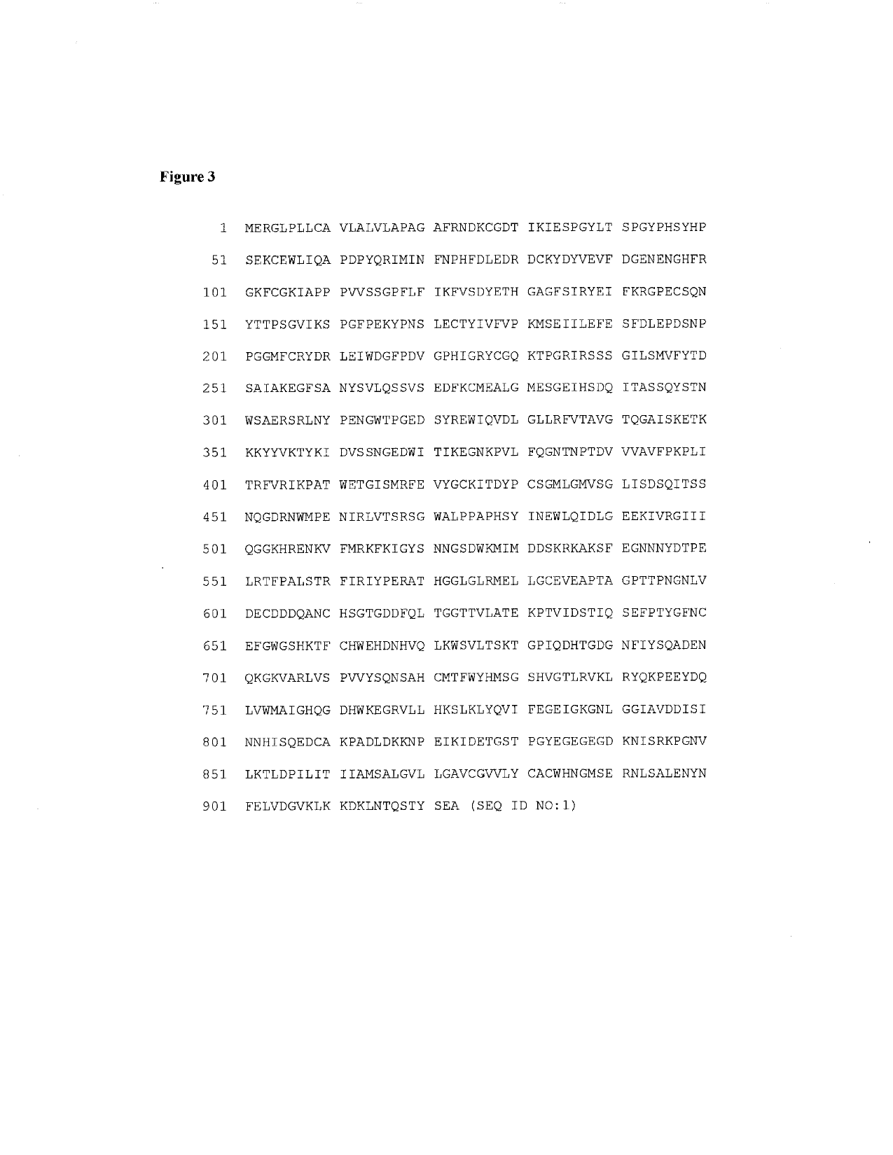

In the context of the present invention, “neuropilin” refers to the neuropilin-1 protein, a type-I membrane protein also known as NRP-1, and exemplified by the amino acid sequence SEQ ID NO:1, shown in FIG. 3 (The NRP-1 precursor amino acid sequence is also available under UniProt accession number 014786). As used herein, “neuropilin” may also refer to neuropilin-2 (also known as NRP-2), which shares approximately 44% homology to NRP-1 as known in the art. Accordingly, the methods of the invention do not distinguish between NRP-1 and NRP-2. In the context of the present invention, the term “neuropilin” also encompasses homologs, variants and isoforms of NRP-1 and/or NRP-2, so long as said homologs, variants and isoforms are specifically recognized by one or more anti-neuropilin antibodies as described herein and/or as known in the art. The term, “neuropilin” further encompasses proteins having at least 85%, at least 90% or at least 95% homology to the amino acid sequence of SEQ ID NO:1, or to the sequence of one or more of a NRP-1 and/or NRP-2 homologue, variant and isoform, including splice isoforms, as well as fragments of the sequences, provided that the variant proteins (including isoforms), homologous proteins and/or fragments are recognized by one or more NRP-1 and/or NRP-2 specific antibodies, such as clone 446915 available from R&D Systems, Inc. (Minneapolis, Minn., U.S.A.), that available as catalog number sc-5307 from Santa Cruz Biotechnology, Inc. (Santa Cruz, Calif., U.S.A.) or that are otherwise known in the art.

Accordingly, the present invention encompasses the determination of expression levels of proteins including, but not limited to, the amino acid sequences as described herein. In certain aspects, the invention encompasses the detection of homologues, variants and isoforms of neuropilin; said isoforms or variants may, inter alia, comprise allelic variants or splice variants. Also envisaged is the detection of proteins that are homologous to neuropilin as herein described, or a fragment thereof, e.g., having at least 60%, 70%, 80%, 90%, 95%, 96%, 97%, 98% or 99% sequence identity to the amino acid sequence of SEQ ID NO:1 or a fragment thereof. Alternatively or additionally, the present invention encompasses detection of the expression levels of proteins encoded by nucleic acid sequences, or fragments thereof, that are at least at least 60%, 70%, 80%, 90%, 95%, 96%, 97%, 98% or 99% identical to a nucleic acid sequence encoding SEQ ID NO:1 or a fragment, variant or isoform thereof. In this context, the term “variant” means that the neuropilin amino acid sequence, or the nucleic acid sequence encoding said amino acid sequence, differs from the distinct sequences identified by SEQ ID NO:1 and/or available under the above-identified UniProt Accession numbers, by mutations, e.g., deletion, additions, substitutions, inversions etc. In addition, the term “homologue” references molecules having at least 60%, more preferably at least 80% and most preferably at least 90% sequence identity to one or more of the polypeptides as shown in SEQ ID NOs:1 or (a) fragment(s) thereof.

In order to determine whether an amino acid or nucleic acid sequence has a certain degree of identity to an amino acid or nucleic acid sequence as herein described, the skilled person can use means and methods well known in the art, e.g. alignments, either manually or by using computer programs known in the art or described herein.

In accordance with the present invention, the term “identical” or “percent identity” in the context of two or more or amino acid or nucleic acid sequences, refers to two or more sequences or subsequences that are the same, or that have a specified percentage of amino acid residues or nucleotides that are the same (e.g., 60% or 65% identity, preferably, 70-95% identity, more preferably at least 95% identity with the amino acid sequences of, e.g., SEQ ID NO:1), when compared and aligned for maximum correspondence over a window of comparison, or over a designated region as measured using a sequence comparison algorithm as known in the art, or by manual alignment and visual inspection. Sequences having, for example, 60% to 95% or greater sequence identity are considered to be substantially identical. Such a definition also applies to the complement of a test sequence. Preferably the described identity exists over a region that is at least about 15 to 25 amino acids or nucleotides in length, more preferably, over a region that is about 50 to 100 amino acids or nucleotides in length. Those having skill in the art will know how to determine percent identity between/among sequences using, for example, algorithms such as those based on CLUSTALW computer program (Thompson Nucl. Acids Res. 2 (1994), 4673-4680) or FASTDB (Brutlag Comp. App. Biosci. 6 (1990), 237-245), as known in the art.

Although the FASTDB algorithm typically does not consider internal non-matching deletions or additions in sequences, i.e., gaps, in its calculation, this can be corrected manually to avoid an overestimation of the % identity. CLUSTALW, however, does take sequence gaps into account in its identity calculations. Also available to those having skill in this art are the BLAST (Basic Local Alignment Search Tool) and BLAST 2.0 algorithms (Altschul, 1997, Nucl. Acids Res. 25:3389-3402; Altschul, 1993 J. Mol. Evol. 36:290-300; Altschul, 1990, J. Mol. Biol. 215:403-410). The BLASTN program for nucleic acid sequences uses as defaults a word length (W) of 11, an expectation (E) of 10, M=5, N=4, and a comparison of both strands. For amino acid sequences, the BLASTP program uses as defaults a wordlength (W) of 3, and an expectation (E) of 10. The BLOSUM62 scoring matrix (Henikoff (1989) PNAS 89:10915) uses alignments (B) of 50, expectation (E) of 10, M=5, N=4, and a comparison of both strands.

BLAST algorithms, as discussed above, produce alignments of both amino and nucleotide sequences to determine sequence similarity. Because of the local nature of the alignments, BLAST is especially useful in determining exact matches or in identifying similar sequences. The fundamental unit of BLAST algorithm output is the High-scoring Segment Pair (HSP). An HSP consists of two sequence fragments of arbitrary but equal lengths whose alignment is locally maximal and for which the alignment score meets or exceeds a threshold or cut-off score set by the user. The BLAST approach is to look for HSPs between a query sequence and a database sequence, to evaluate the statistical significance of any matches found, and to report only those matches which satisfy the user-selected threshold of significance. The parameter E establishes the statistically significant threshold for reporting database sequence matches. E is interpreted as the upper bound of the expected frequency of chance occurrence of an HSP (or set of HSPs) within the context of the entire database search. Any database sequence whose match satisfies E is reported in the program output.

Analogous computer techniques using BLAST may be used to search for identical or related molecules in protein or nucleotide databases such as GenBank or EMBL. This analysis is much faster than multiple membrane-based hybridizations. In addition, the sensitivity of the computer search can be modified to determine whether any particular match is categorized as exact or similar. The basis of the search is the product score which is defined as:

% sequence identity×% maximum BLAST score/100

and takes into account both the degree of similarity between two sequences and the length of the sequence match. For example, with a product score of 40, the match will be exact within a 1-2% error; and at 70, the match will be exact. Similar molecules are usually identified by selecting those which show product scores between 15 and 40, although lower scores may identify related molecules. Another example for a program capable of generating sequence alignments is the CLUSTALW computer program (Thompson, 1994, Nucl. Acids Res. 2:4673-4680) or FASTDB (Brutlag, 1990, Comp. App. Biosci. 6:237-245), as is known in the art.

In accordance with the present invention, it was surprisingly discovered in the AVAGAST population (see, e.g., Kang et al., 2010, J. Clin. Oncol., 28:18s (suppl. abstr. LBA4007)) that a greater bevacizumab treatment effect was associated with lower tumor specific neuropilin expression. Specifically, relatively lower tumor specific neuropilin expression was associated with improved overall survival and/or improved progression free survival in patients receiving bevacizumab in addition to the chemotherapeutic regimen.

The expression level of neuropilin (e.g., NRP-1, NRP-2, or a variant, homologue, truncation or fragment thereof) may be assessed by any method known in the art suitable for determination of specific protein levels in a patient sample and is preferably determined by an immunohistochemical (“IHC”) method employing antibodies specific for neuropilin. Such methods are well known and routinely implemented in the art and corresponding commercial antibodies and/or kits are readily available. For example, commercially available antibodies specific for neuropilin as described and defined herein can be obtained from R&D Systems, Inc. (Minneapolis, Minn., U.S.A.) as clone 446915 and from Santa Cruz Biotechnology, Inc. (Santa Cruz, Calif., U.S.A.) as catalog number sc-5307. Preferably, the expression levels of the marker/indicator proteins of the invention are assessed using the reagents and/or protocol recommendations of the antibody or kit manufacturer. The skilled person will also be aware of further means for determining the expression level of neuropilin by IHC methods. Therefore, the expression level of neuropilin and/or other markers/indicators as known in the art can be routinely and reproducibly determined by a person skilled in the art without undue burden. However, to ensure accurate and reproducible results, the invention also encompasses the testing of patient samples in a specialized laboratory that can ensure the validation of testing procedures.

Preferably, the expression level of neuropilin is assessed in a biological sample that contains or is suspected to contain cancer cells and is determined in a tumor-specific manner. The sample may comprise both cancer cells, i.e., tumor cells, and non-cancerous cells, e.g., endothelial or non-malignant cells. In some aspects, determination of the tumor-specific expression of neuropilin relates to the determination of the expression levels of exclusively cancer cells as opposed to other cell types, e.g., endothelial or non-cancerous/non-malignant cells, that may be present in the tumor sample. In other aspects, determination of the tumor-specific expression of neuropilin relates to the determination of expression levels of cancer cells as well as any other cell-type, e.g., endothelial cells, that may be present in the tumor sample. The skilled artisan, e.g., a pathologist, can readily discern cancer cells from non-cancerous cells, e.g., endothelial cells. The sample may be a gastric tissue resection or a gastric tissue biopsy obtained from a patient suffering from, suspected to suffer from or diagnosed with gastric cancer, in particular, adenocarcinoma of the stomach or GEJ. The sample may also be a resection or biopsy of a metastatic lesion obtained from a patient suffering from, suspected to suffer from or diagnosed with gastric cancer, in particular, adenocarcinoma of the stomach or GEJ. Preferably, the sample is a sample of stomach tissue or tissue of the gastro-esophageal junction, or a resection or biopsy of an adenocarcinoma of the stomach or gastro-esophageal junction. The sample may also be a sample of a known or suspected metastatic gastric cancer lesion or section, or a blood sample, e.g., a peripheral blood sample, known or suspected to comprise circulating cancer cells, e.g., gastric cancer cells. The analysis of the sample according to the methods of the invention may be manual, as performed by the skilled artisan, e.g., a pathologist, as is known in the art, or may be automated using commercially available software designed for the processing and analysis of pathology images, e.g., for analysis in tissue biopsies or resections (e.g., MIRAX SCAN, Carl Zeiss AG, Jena, Germany). Methods of obtaining biological samples including tissue resections, biopsies and body fluids, e.g., blood samples comprising cancer/tumor cells, are well known in the art.

In the context of the present invention, bevacizumab is to be administered in addition to or as a co-therapy or co-treatment with one or more chemotherapeutic agents administered as part of standard chemotherapy regimen as known in the art. Examples of such chemotherapeutic agents include 5-fluorouracil, leucovorin, irinotecan, gemcitabine-erlotinib, capecitabine and platinum-based chemotherapeutic agents, such as paclitaxel, carboplatin, cisplatin and oxaliplatin. As demonstrated in the appended examples, the addition of bevacizumab to capecitabine- or 5-fluorouracil-based chemotherapeutic regimens effected an increase in progression free survival and correlated with overall survival in the gastric cancer patients and/or patient population defined and selected according to the expression level of neuropilin, in particular, having lower expression of neuropilin in tumor samples relative to control levels established in similarly situated patients.

Bevacizumab may be combined with a capecitabine- or 5-fluorouracil-based chemotherapy regimen. The selection between capecitabine and 5-fluorouracil is best determined by the treating physician based on standards well established in the art. Examples of capecitabine-based chemotherapy regimens include the combination of capecitabine (or 5-fluorouracil) administered in combination with cisplatin. A typical cycle of capecitabine/cisplatin therapy may be capecitabine administered at a dose of 1000 mg/m² orally twice daily (bid) over days 1 to 14, followed by 1 week rest, and cisplatin at a dose of 80 mg/m² administered i.v. as a 2 hour infusion on day 1 of the cycle with hyper-hydration and pre-medication (steroids and anti-emetics; 3×/week); the cisplatin and capecitabine cycle is continued until disease progression or unmanageable toxicity, with cisplatin administration limited to a maximum of 6 cycles. Accordingly, in certain aspects of the invention, the patient identified according to the methods herein is treated with bevacizumab in combination with capecitabine/cisplatin. Common modes of administration of bevacizumab include parenteral administration as a bolus dose or as an infusion over a set period of time, e.g., administration of the total daily dose over 10 min., 20 min., 30 min., 40 min., 50 min., 60 min., 75 min., 90 min., 105 min., 120 min., 3 hr., 4 hr., 5 hr. or 6 hr. For example, 7.5 mg/kg of bevacizumab (Avastin®) may be administered to patients with gastric cancer as an intravenous infusion over 15 to 30 minutes on day 1 of every capecitabine cycle as described above. The skilled person will recognize that further modes of administration of bevacizumab are encompassed by the invention as determined by the specific patient and chemotherapy regimen, and that the specific mode of administration and therapeutic dosage are best determined by the treating physician according to methods known in the art.

The patients selected according to the methods of the present invention are treated with bevacizumab in combination with a chemotherapy regimen, and may be further treated with one or more additional anti-cancer therapies. In certain aspects, the one or more additional anti-cancer therapy is radiation.

In preferred embodiments, the sample obtained from the patient is collected prior to beginning any other chemotherapeutic regimen or therapy, e.g., therapy for the treatment of cancer or the management or amelioration of a symptom thereof. Therefore, in preferred embodiments, the sample is collected before the administration of chemotherapeutics or the start of a chemotherapy regimen.

The present invention also relates to a diagnostic composition or kit comprising oligonucleotides or polypeptides suitable for the determination of the tumor specific expression level of neuropilin. As detailed herein, oligonucleotides such as DNA, RNA or mixtures of DNA and RNA probes may be of use in detecting mRNA levels of the marker/indicator proteins, in particular, neuropilin, while polypeptides may be of use in directly detecting protein levels of the marker/indicator proteins via specific protein-protein interaction. In preferred aspects of the invention, the polypeptides encompassed as probes for the expression levels of neuropilin, and included in the kits or diagnostic compositions described herein, are antibodies specific for neuropilin, or specific for homologues, variants and/or truncations thereof.

Accordingly, a further embodiment of the present invention provides a kit useful for carrying out the methods herein described, comprising oligonucleotides or polypeptides capable of determining the expression level of neuropilin. Preferably, the oligonucleotides comprise primers and/or probes specific for the mRNA encoding neuropilin as defined and described herein, and the polypeptides comprise proteins capable of specific interaction with neuropilin, e.g., marker/indicator specific antibodies or antibody fragments.

In a further embodiment, the present invention provides the use of bevacizumab for improving treatment effect in a patient suffering from gastric cancer, in particular, adenocarcinoma of the stomach or GEJ, comprising the following steps:
(a) determining the expression level of neuropilin in a patient sample; and
(b) administering bevacizumab in combination with a chemotherapy regimen to the patient having a decreased level of neuropilin relative to control levels determined in patients suffering from gastric cancer, in particular, adenocarcinoma of the stomach or GEJ.
   The improved treatment effect may be improved overall survival or improved progression free survival.

As documented in the appended examples, the present invention solves the identified technical problem in that it could surprisingly be shown that the expression level of neuropilin in a given patient, relative to a control level determined in patients diagnosed with gastric cancer, in particular, adenocarcinoma of the stomach or GEJ, correlate with treatment effect in patients administered bevacizumab in combination with a capecitabine- or 5-fluorouracil-based chemotherapy regimen.

The phrase “responsive to” in the context of the present invention indicates that a subject/patient suffering from, suspected to suffer or prone to suffer from, or diagnosed with gastric cancer, in particular, adenocarcinoma of the stomach or GEJ, shows a response to a chemotherapy regimen comprising the addition of bevacizumab. A skilled person will readily be in a position to determine whether a person treated with bevacizumab according to the methods of the invention shows a response. For example, a response may be reflected by decreased suffering from gastric cancer, such as a diminished and/or halted tumor growth, reduction of the size of a tumor, and/or amelioration of one or more symptoms of gastric cancer, e.g., gastrointestinal bleeding, pain, anemia. Preferably, the response may be reflected by decreased or diminished indices of the metastatic conversion of gastric cancer, e.g., the prevention of the formation of metastases or a reduction of number or size of metastases,

The phrase “sensitive to” in the context of the present invention indicates that a subject/patient suffering from, suspected to suffer or prone to suffer from, or diagnosed, with gastric cancer, in particular, adenocarcinoma of the stomach or GEJ, shows in some way a positive reaction to treatment with bevacizumab in combination with a chemotherapy regimen. The reaction of the patient may be less pronounced when compared to a patient “responsive to” as described hereinabove. For example, the patient may experience less suffering associated with the disease, though no reduction in tumor growth or metastatic indicator may be measured, and/or the reaction of the patient to the bevacizumab in combination with the chemotherapy regimen may be only of a transient nature, i.e., the growth of (a) tumor and/or (a) metastasis(es) may only be temporarily reduced or halted.

The phrase “a patient suffering from” in accordance with the invention refers to a patient showing clinical signs of gastric cancer, in particular, adenocarcinoma of the stomach or GEJ. The gastric cancer may be metastatic, inoperable and/or locally advanced adenocarcinoma of the stomach or gastro-esophageal junction (“GEJ”). The phrase “being susceptible to” or “being prone to,” in the context of gastric cancer, refers to an indication disease in a patient based on, e.g., a possible genetic predisposition, a pre- or eventual exposure to hazardous and/or carcinogenic compounds, or exposure to carcinogenic physical hazards, such as radiation.

The phrase “treatment effect” in the context of the present invention encompasses the phrases “progression free survival” and “overall survival”.

The phrase “progression-free survival” in the context of the present invention refers to the length of time during and after treatment during which, according to the assessment of the treating physician or investigator, the patient's disease does not become worse, i.e., does not progress. As the skilled person will appreciate, a patient's progression-free survival is improved or enhanced if the patient experiences a longer length of time during which the disease does not progress as compared to the average or mean progression free survival time of a control group of similarly situated patients.

The phrase “overall survival” in the context of the present invention refers to the average survival of the patient within a patient group. As the skilled person will appreciate, a patient's overall survival is improved or enhanced, if the patient belongs to a subgroup of patients that has a statistically significant longer mean survival time as compared to another subgroup of patients. Improved overall survival may be evident in one or more subgroups of patients but not apparent when the patient population is analysed as a whole.

The terms “administration” or “administering” as used herein mean the administration of an angiogenesis inhibitor, e.g., bevacizumab (Avastin®), and/or a pharmaceutical composition/treatment regimen comprising an angiogenesis inhibitor, e.g., bevacizumab (Avastin®), to a patient in need of such treatment or medical intervention by any suitable means known in the art for administration of a therapeutic antibody. Nonlimiting routes of administration include by oral, intravenous, intraperitoneal, subcutaneous, intramuscular, topical, intradermal, intranasal or intrabronchial administration (for example as effected by inhalation). Particularly preferred in context of this invention is parenteral administration, e.g., intravenous administration. With respect to bevacizumab (Avastin®) for the treatment of colorectal cancer, the preferred dosages according to the EMEA are 5 mg/kg or 10 mg/kg of body weight given once every 2 weeks or 7.5 mg/kg or 15 mg/kg of body weight given once every 3 weeks (for details see http://www.emea.europa.eu/humandocs/PDFs/EPAR/avastin/emea-combined-h582en.pdf).

The term “antibody” is herein used in the broadest sense and includes, but is not limited to, monoclonal and polyclonal antibodies, multispecific antibodies (e.g., bispecific antibodies), chimeric antibodies, CDR grafted antibodies, humanized antibodies, camelized antibodies, single chain antibodies and antibody fragments and fragment constructs, e.g., F(ab′)₂ fragments, Fab-fragments, Fv-fragments, single chain Fv-fragments (scFvs), bispecific scFvs, diabodies, single domain antibodies (dAbs) and minibodies, which exhibit the desired biological activity, in particular, specific binding to one or more of VEGFA, HER2, neuropilin and CD31, or to homologues, variants, fragments and/or isoforms thereof.

As used herein “chemotherapeutic agent” includes any active agent that can provide an anticancer therapeutic effect and may be a chemical agent or a biological agent, in particular, that are capable of interfering with cancer or tumor cells. Preferred active agents are those that act as anti-neoplastic (chemotoxic or chemostatic) agents which inhibit or prevent the development, maturation or proliferation of malignant cells. Nonlimiting examples of chemotherapeutic agents include alkylating agents such as nitrogen mustards (e.g., mechlorethamine, cyclophosphamide, ifosfamide, melphalan and chlorambucil), nitrosoureas (e.g., carmustine (BCNU), lomustine (CCNU), and semustine (methyl-CCNU)), ethylenimines/methylmelamines (e.g., thriethylenemelamine (TEM), triethylene, thiophosphoramide (thiotepa), hexamethylmelamine (HMM, altretamine)), alkyl sulfonates (e.g., busulfan), and triazines (e.g., dacarbazine (DTIC)); antimetabolites such as folic acid analogs (e.g., methotrexate, trimetrexate), pyrimidine analogs (e.g., 5-fluorouracil, fluorodeoxyuridine, gemcitabine, cytosine arabinoside (AraC, cytarabine), 5-azacytidine, 2,2′-difluorodeoxycytidine, and pyrimidine analog prodrugs, e.g., capecitabine), purine analogs (e.g., 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA)); antimitotic drugs developed from natural products (e.g., paclitaxel, vinca alkaloids (e.g., vinblastine (VLB), vincristine, and vinorelbine), taxotere, estramustine, and estramustine phosphate), epipodophylotoxins (e.g., etoposide, teniposide), antibiotics (e.g., actimomycin D, daunomycin (rubidomycin), doxorubicin, mitoxantrone, idarubicin, bleomycins, plicamycin (mithramycin), mitomycinC, actinomycin), enzymes (e.g., L-asparaginase), and biological response modifiers (e.g., interferon-alpha, IL-2, G-CSF, GM-CSF); miscellaneous agents including platinum coordination complexes (e.g., cisplatin, carboplatin), anthracenediones (e.g., mitoxantrone), substituted urea (i.e., hydroxyurea), methylhydrazine derivatives (e.g., N-methylhydrazine (MIH), procarbazine), adrenocortical suppressants (e.g., mitotane (o,p′-DDD), aminoglutethimide); hormones and antagonists including adrenocorticosteroid antagonists (e.g., prednisone and equivalents, dexamethasone, aminoglutethimide), progestins (e.g., hydroxyprogesterone caproate, medroxyprogesterone acetate, megestrol acetate), estrogens (e.g., diethylstilbestrol, ethinyl estradiol and equivalents thereof); antiestrogens (e.g., tamoxifen), androgens (e.g., testosterone propionate, fluoxymesterone and equivalents thereof), antiandrogens (e.g., flutamide, gonadotropin-releasing hormone analogs, leuprolide) and non-steroidal antiandrogens (e.g., flutamide).

In the context of the present invention, “homology” with reference to an amino acid sequence is understood to refer to a sequence identity of at least 80%, particularly an identity of at least 85%, preferably at least 90% and still more preferably at least 95% over the full length of the sequence as defined by the SEQ ID NO(s) provided herein. In the context of this invention, a skilled person would understand that homology covers further allelic variation(s) of the marker/indicator proteins in different populations and ethnic groups.

As used herein, the term “polypeptide” relates to a peptide, a protein, an oligopeptide or a polypeptide which encompasses amino acid chains of a given length, wherein the amino acid residues are linked by covalent peptide bonds. However, peptidomimetics of such proteins/polypeptides are also encompassed by the invention wherein amino acid(s) and/or peptide bond(s) have been replaced by functional analogs, e.g., an amino acid residue other than one of the 20 gene-encoded amino acids, e.g., selenocysteine. Peptides, oligopeptides and proteins may be termed polypeptides. The terms polypeptide and protein are used interchangeably herein. The term polypeptide also refers to, and does not exclude, modifications of the polypeptide, e.g., glycosylation, acetylation, phosphorylation and the like. Such modifications are well described in basic texts and in more detailed monographs, as well as in a voluminous research literature.

The terms “treating” and “treatment” as used herein refer to remediation of, improvement of, lessening of the severity of, or reduction in the time course of the disease or any parameter or symptom thereof. Preferably said patient is a human patient and the disease to be treated is a gastric cancer, in particular, adenocarcinoma of the stomach or GEJ. The terms “assessing” or “assessment” of such a patient relates to methods of determining the expression levels of neuropilin, and/or for selecting such patients based on the expression levels of such marker/indicator proteins relative to control levels established in patients diagnosed with metastatic colorectal cancer.

In addition to the methods described above, the invention also encompasses further immunohistochemical methods for assessing the expression level of neuropilin, such as by Western blotting and ELISA-based detection. As is understood in the art, the expression level of the marker/indicator proteins of the invention may also be assessed at the mRNA level by any suitable method known in the art, such as Northern blotting, real time PCR, and RT PCR. Immunohistochemical- and mRNA-based detection methods and systems are well known in the art and can be deduced from standard textbooks, such as Lottspeich (Bioanalytik, Spektrum Akademisher Verlag, 1998) or Sambrook and Russell (Molecular Cloning: A Laboratory Manual, CSH Press, Cold Spring Harbor, N.Y., U.S.A., 2001). The described methods are of particular use for determining the expression level, e.g., tumor specific expression level, of neuropilin in a patient or group of patients relative to control levels established in a similarly situated population, e.g., suffering from or diagnosed with gastric cancer, in particular, adenocarcinoma of the stomach or GEJ.

The expression level of neuropilin can also be determined on the protein level by taking advantage of immunoagglutination, immunoprecipitation (e.g., immunodiffusion, immunelectrophoresis, immune fixation), western blotting techniques (e.g., (in situ) immuno histochemistry, (in situ) immuno cytochemistry, affinity chromatography, enzyme immunoassays), and the like. Amounts of purified polypeptide in solution may also be determined by physical methods, e.g. photometry. Methods of quantifying a particular polypeptide in a mixture usually rely on specific binding, e.g., of antibodies. Specific detection and quantitation methods exploiting the specificity of antibodies comprise for example immunohistochemistry (in situ). For example, the concentration/amount of the marker/indicator proteins of the present invention (e.g., NRP-1, NRP-2 and/or a variant, homolog or truncation thereof) in a cell or tissue may be determined by enzyme linked-immunosorbent assay (ELISA). Alternatively, Western Blot analysis or immunohistochemical staining can be performed. Western blotting combines separation of a mixture of proteins by electrophoresis and specific detection with antibodies. Electrophoresis may be multi-dimensional such as 2D electrophoresis. Usually, polypeptides are separated in 2D electrophoresis by their apparent molecular weight along one dimension and by their isoelectric point along the other direction.

As mentioned above, the decreased expression of the marker/indicator proteins according to the present invention may also be reflected in a decreased expression of the corresponding gene(s) for neuropilin (as described and defined herein). Therefore, a quantitative assessment of the gene product prior to translation (e.g. spliced, unspliced or partially spliced mRNA) can be performed in order to evaluate the expression of the corresponding gene(s). The person skilled in the art is aware of standard methods to be used in this context or may deduce these methods from standard textbooks (e.g. Sambrook, 2001, loc. cit.). For example, quantitative data on the respective concentration/amounts of mRNA encoding neuropilin can be obtained by Northern Blot, Real Time PCR and the like.

In a further aspect of the invention, the kit of the invention may advantageously be used for carrying out a method of the invention and could be, inter alia, employed in a variety of applications, e.g., in the diagnostic field or as a research tool. The parts of the kit of the invention can be packaged individually in vials or in combination in containers or multicontainer units. Manufacture of the kit follows preferably standard procedures which are known to the person skilled in the art. The kit or diagnostic compositions may be used for detection of the expression level of neuropilin (as defined and described herein) in accordance with the herein-described methods of the invention, employing, for example, immunohistochemical techniques.

Although exemplified by the use of bevacizumab, the invention encompasses the use of other angiogenesis inhibitors known in the art for use in combination with standard chemotherapy regimens. The terms “angiogenesis inhibitor” as used herein refers to all agents that alter angiogenesis (e.g. the process of forming blood vessels) and includes agents that block the formation of and/or halt or slow the growth of blood vessels. Nonlimiting examples of angiogenesis inhibitors include, in addition to bevacizumab, pegaptanib, sunitinib, sorafenib and vatalanib. Preferably, the angiogenesis inhibitor for use in accordance with the methods of the present invention is bevacizumab. As used herein, the term “bevacizumab” encompass all corresponding anti-VEGF antibodies or anti-VEGF antibody fragments, that fulfil the requirements necessary for obtaining a marketing authorization as an identical or biosimilar product in a country or territory selected from the group of countries consisting of the USA, Europe and Japan.

For use in the detection methods described herein, the skilled person has the ability to label the polypeptides or oligonucleotides encompassed by the present invention. As routinely practiced in the art, hybridization probes for use in detecting mRNA levels and/or antibodies or antibody fragments for use in IHC methods can be labelled and visualized according to standard methods known in the art. Nonlimiting examples of commonly used systems include the use of radiolabels, enzyme labels, fluorescent tags, biotin-avidin complexes, chemiluminescence, and the like.

The person skilled in the art, for example, the attending physician, is readily in a position to administer the bevacizumab in combination with a chemotherapy regimen to the patient/patient group as selected and defined herein. In certain contexts, the attending physician may modify, change or amend the administration schemes for the bevacizumab and the chemotherapy regimen in accordance with his/her professional experience. Therefore, in certain aspects of the present invention, a method is provided for the treatment or improving treatment effect (i.e., the progression-free or overall survival) in a patient suffering from or suspected to suffer from gastric cancer with bevacizumab in combination with a chemotherapy regimen, whereby said patient/patient group is characterized in the assessment of a biological sample (in particular a gastric tissue resection, gastric tissue biopsy and/or metastatic lesion), said sample exhibiting a decreased expression level of neuropilin, relative to control levels established in patients suffering from and/or diagnosed with gastric cancer, in particular, adenocarcinoma of the stomach or GEJ. The present invention also provides for the use of bevacizumab in the preparation of pharmaceutical composition for the treatment of a patient suffering from or suspected to suffer from gastric cancer, in particular, adenocarcinoma of the stomach or GEJ, wherein the patients are selected or characterized by the herein disclosed protein marker/indicator status (i.e., a decreased expression level of neuropilin relative to control levels established in patients suffering from gastric cancer, in particular, adenocarcinoma of the stomach or GEJ).

The figures show:

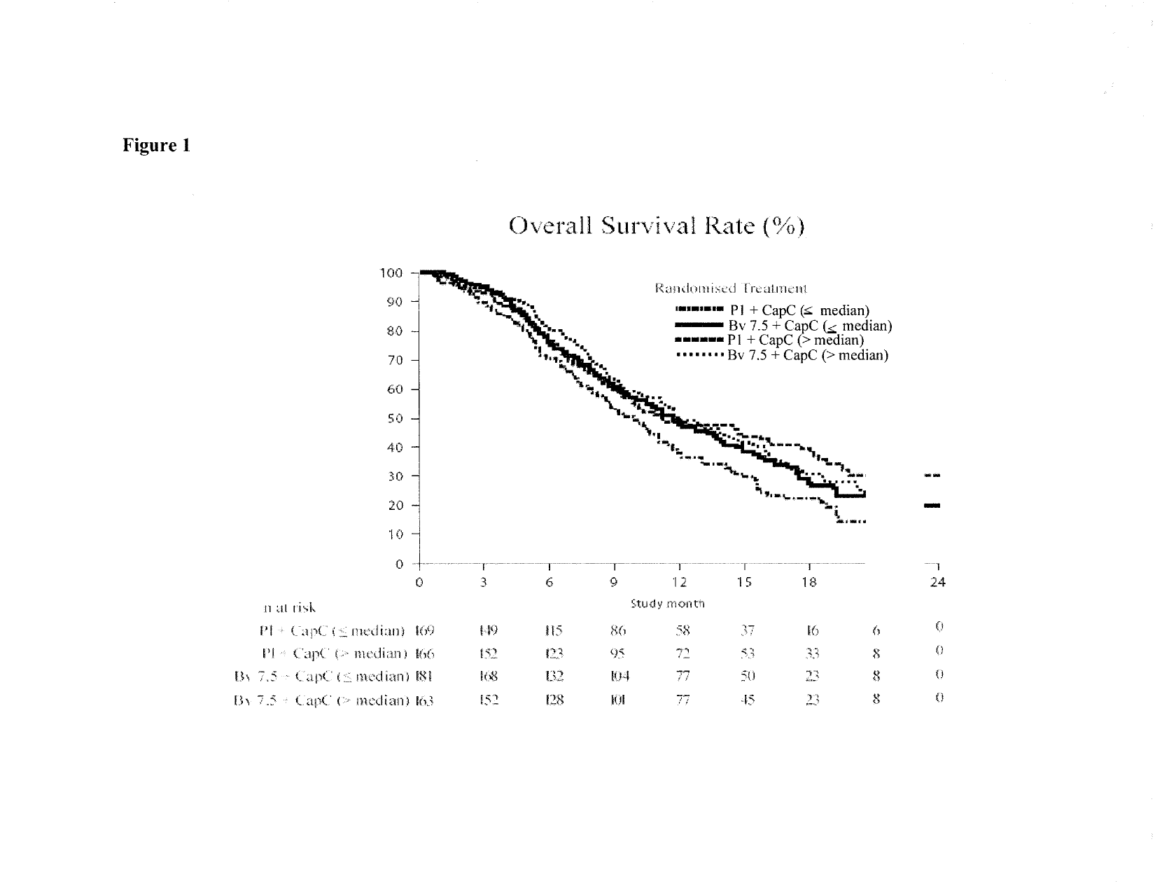

FIG. 1: Correlation of neuropilin expression with overall survival (median cut-off). Solid line, placebo, chemotherapy and neuropilin expression above median; Long-dashed line, bevacizumab therapy, chemotherapy and biomarker expression above median; Medium-dashed line, bevacizumab therapy, chemotherapy and biomarker expression below or equal to median; Short-dashed line, placebo, chemotherapy and biomarker expression below or equal to median.

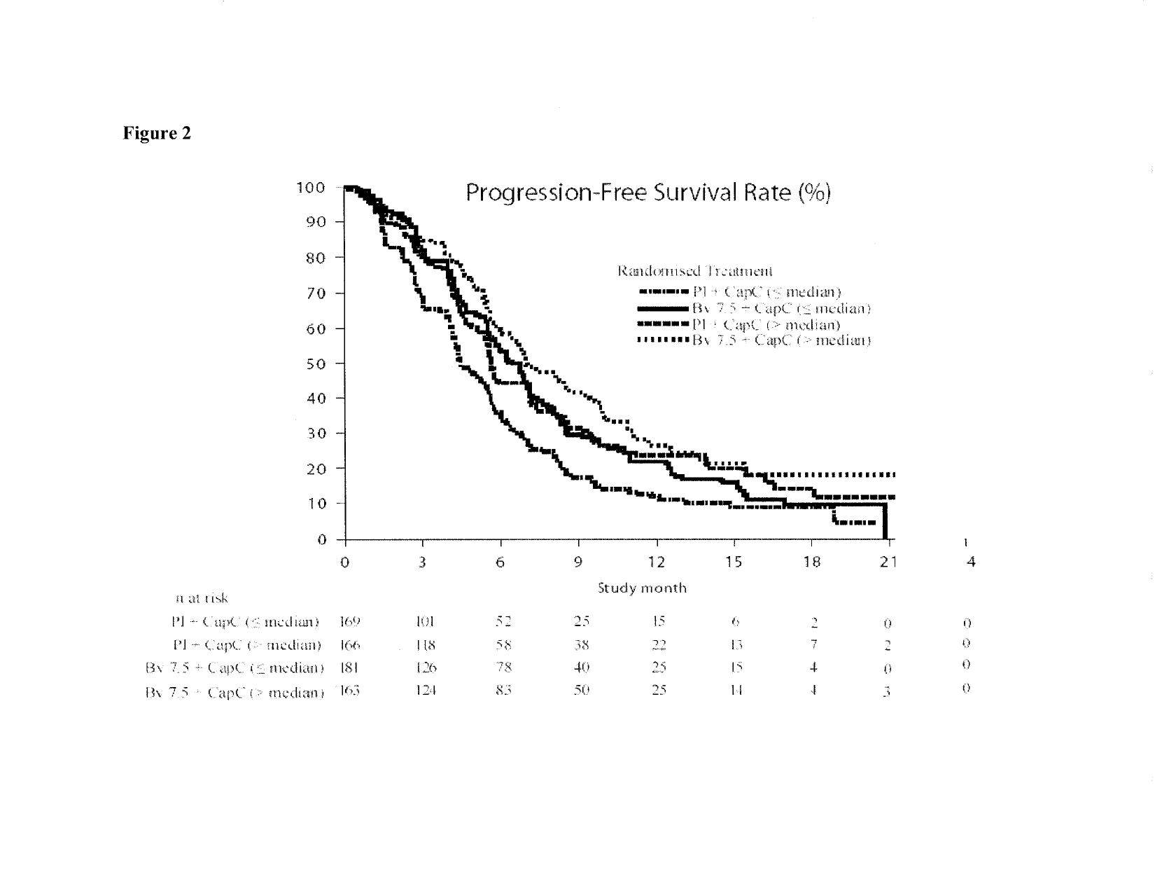

FIG. 2: Correlation of neuropilin expression with time to progression or death (median cut-off). Solid line, placebo, chemotherapy and neuropilin expression above median; Long-dashed line, bevacizumab therapy, chemotherapy and biomarker expression above median; Medium-dashed line, bevacizumab therapy, chemotherapy and biomarker expression below or equal to median; Short-dashed line, placebo, chemotherapy and biomarker expression below or equal to median.

FIG. 3: SEQ ID NO:1, representative amino acid sequence of neuropilin-1.

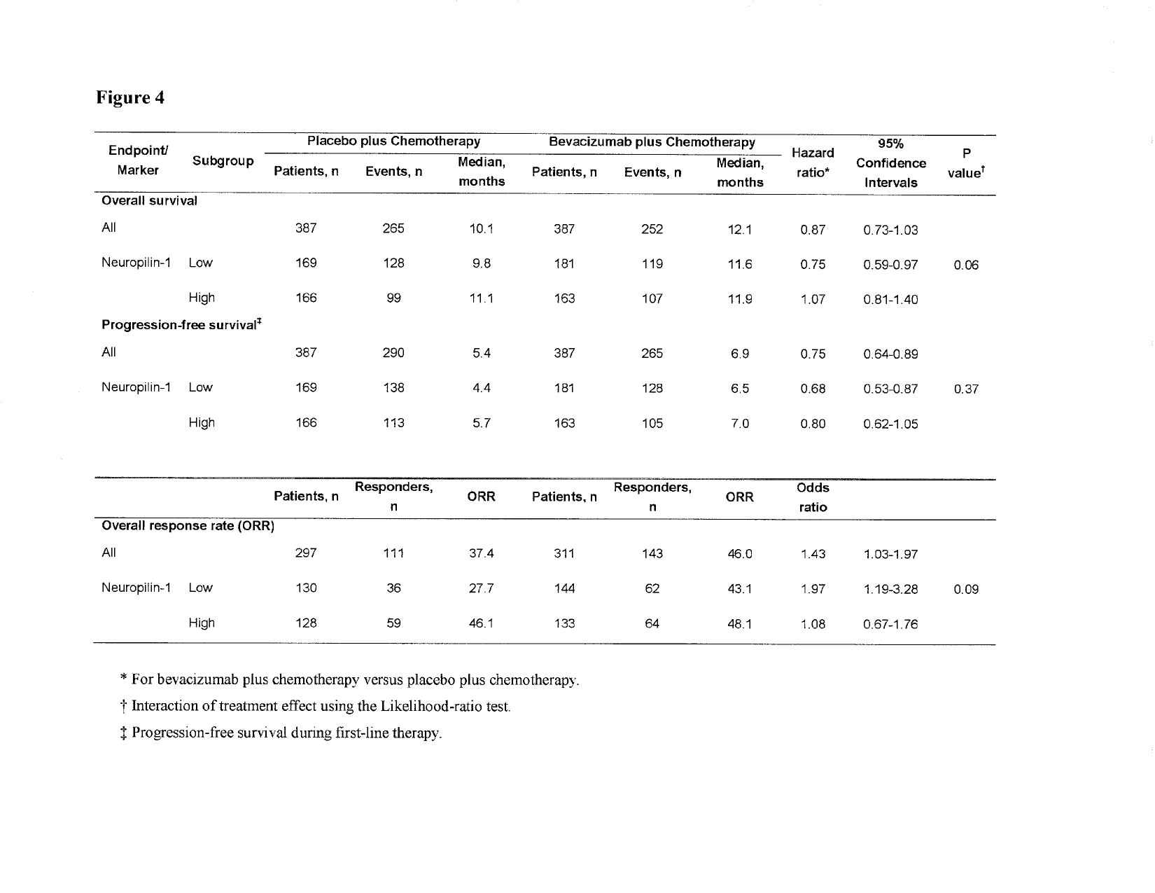

FIG. 4: Correlation between neuropilin expression with overall survival, time to progression or death, and overall response rate (ORR).

### EXAMPLES

Tissue samples were collected from patients participating a randomized phase-III study comparing the results of adding bevacizumab to first-line capecitabine (5-fluoruracil was allowed if capecitabine was contraindicated)/cisplatin combination chemotherapy regimens for the treatment of metastatic or inoperable, locally advanced adenocarcinoma of the stomach or GEJ (the AVAGAST study, see, Kang et al., 2010, J. Clin. Oncol., 28:18s (suppl. abstr. LBA4007) (“Kang”)). An investigation of the status of biomarkers related to angiogenesis and tumorigenesis revealed that a decreased expression level of neuropilin relative to a control level determined in the entire patient population indicated improved overall survival and/or progression free survival.

Patients and Immunohistochemical Methods

A total of 774 patients participated in the AVAGAST study, and tumor samples from between 629 and 727 of the participants were available for biomarker analysis, dependent on the specific biomarker. Treatment arms were balanced. Approximately 95% of the patients were metastatic. Approximately ⅔ of the patients were male, 49% were from Asia/Pacific, 32% were from Europe and 19% were from the Americas (see, Kang).

Tissue samples were available as tissue blocks or as previously prepared slides. Immunohistochemical analysis was performed on 5 μm sections of formalin-fixed paraffin-embedded tissue samples (for blocks) or on the previously prepared slides. After deparaffinization and rehydration, antigen retrieval was performed by citrate pH 6.0 buffer at 95° C. for 30 minutes in a PT module or CC1 buffer in the Benchmark-XT (Ventana, Tucson, Ariz., USA).

Initial biomarkers, including neuropilin, were selected for immunohistochemical analysis based on known tumorigenic and angiogenic activity. In particular, neuropilin was analysed using the anti-human neuropilin murine monoclonal antibody available from Santa Cruz Biotechnology, Inc. (Santa Cruz, Calif., U.S.A) as catalog number sc-5307.

Sections were stained on Autostainer or Benchmark-XT (for VEGFR-1) and primary antibodies were incubated for 1 hour. With specific respect to the Santa Cruz antibody, this anti-neuropilin antibody was used at 1/50 dilution. Binding of the primary antibodies was visualized using the Envision system (DAKO, Glostrup, Denmark) or Ultraview (Ventana, Tucson, Ariz. USA). All sections were counterstained with Mayer's hematoxylin.

Validation reports showing accuracy, specificity, linearity, and precision (reproducibility and repeatability) were produced for each IHC assay. Staining of external control slides and intrinsic control elements was documented.

Statistical Analysis

The overall distribution of biomarkers was described using the H-score for tumor markers. The number of markers examined was limited and each one was supported by a biological rationale; there was no formal correction for multiple testing. The a priori cut-off was used for protein expression level: median (below, above) and quartile (≦25, 25<x≦50, 50<x≦75, >75).

Treatment effects were estimated in subgroups of patients defined by biomarker level. Overall survival (“OS”) and/or progression free survival (“PFS”) was chosen as the primary endpoint; the primary descriptive analysis was performed using subgroup analysis. Test of treatment by biomarker interactions (median cut-off) also provided a secondary analysis.

Results

Tumor Markers

Results of the analysis of the tumor samples for neuropilin are provided in Table 1.

TABLE 1
Neuropilin H scores determined by IHC analysis of AVAGAST samples

| | Pl + CapC | Bv7.5 + CapC | All Patients |
|---|---|---|---|
| | N = 387 | N = 387 | N = 774 |
| n | 335 | 344 | 679 |
| Geometric Mean | 35 | 38 | 36 |
| Arithmetic Mean | 85 | 86 | 86 |
| SE | 3.1 | 3.1 | 2.2 |
| SD | 57.0 | 57.8 | 57.4 |
| Min-Max | 0-210 | 0-250 | 0-250 |
| 25^{th} percentile | 40 | 40 | 40 |
| Median | 90 | 90 | 90 |
| 75^{th} percentile | 120 | 118 | 120 |
| CV (%) | 67 | 67 | 67 |
| Number of Min | 41 | 33 | 74 |
| Number of Max | 1 | 1 | 1 |
| Number with 100% Staining | 118 | 114 | 232 |

The median H score of neuropilin expression used for subsequent analysis was 90, with 25th and 75^{th} percentile scores of 40 and 120, respectively.

Biomarker Correlation with Overall Survival

Hazard ratios were determined for overall survival in patients separated by median or quartile neuropilin H scores.

TABLE 2
Hazard ratios for overall survival in AVAGAST patients
separated by median neuropilin H score

| Neuropilin | | Lower | Hazard Ratio | Upper Confidence |
|---|---|---|---|---|
| H score | N | Confidence Limit | Estimate | Limit |
| <=median | 350 | 0.59 | 0.75 | 0.97 |
| >median | 329 | 0.81 | 1.07 | 1.40 |

TABLE 3
Hazard ratios for overall survival in AVAGAST patients
separated by quartile neuropilin H score

| | | | Hazard | |
|---|---|---|---|---|
| Neuropilin | | Lower | Ratio | Upper Confidence |
| H score | N | Confidence Limit | Estimate | Limit |
| <=P25 | 186 | 0.48 | 0.68 | 0.96 |
| P25 to <=P50 | 164 | 0.57 | 0.83 | 1.19 |
| P50 to <=P75 | 184 | 0.67 | 0.97 | 1.42 |
| >P75 | 145 | 0.79 | 1.19 | 1.78 |

The calculated hazard ratios indicate that overall survival improves in those patients exhibiting relatively decreased tumor specific expression of neuropilin when administered bevacizumab in combination with the standard chemotherapy. In particular, in Table 2, the upper bound of the 95% confidence interval of treatment hazard ratio in the subset of patients with low tumor specific neuropilin expression (≦median) is below 1. This supports the statistical relevance of the treatment effect (overall survival) observed in this sub-group of patients.

A Kaplan-Meier curve correlating bevacizumab treatment and neuropilin expression with respect to overall survival is provided in FIG. 1 (median cut-off). The improvement in overall survival for those patients having relatively low neuropilin expression when bevacizumab is added to the chemotherapy, indicated in the hazard ratios, is also visible in FIG. 5. Median overall survival was improved by 1.8 months in patients with relatively low tumor specific neuropilin expression (≦median) compared to only 0.8 months for patients with tumor specific neuropilin expression above median. The results demonstrate that the treatment effect (overall survival) is improved in the subset of patients with relatively low level of neuropilin.

Biomarker Correlation with Progression Free Survival

Hazard ratios were determined for time to disease progression or death in patients separated by median or quartile neuropilin H scores.

TABLE 4
Hazard ratios for time to disease progression or death
in AVAGAST patients separated by median neuropilin H score

| Neuropilin | | Lower | Hazard Ratio | Upper Confidence |
|---|---|---|---|---|
| H score | N | Confidence Limit | Estimate | Limit |
| <=median | 350 | 0.53 | 0.68 | 0.87 |
| >median | 329 | 0.62 | 0.80 | 1.05 |

TABLE 5
Hazard ratios for time to disease progression or death
in AVAGAST patients separated by quartile neuropilin H score

| | | | Hazard | |
|---|---|---|---|---|
| Neuropilin | | Lower | Ratio | Upper Confidence |
| H score | N | Confidence Limit | Estimate | Limit |
| <=P25 | 178 | 0.51 | 0.71 | 0.98 |
| P25 to <=P50 | 160 | 0.47 | 0.68 | 0.98 |
| P50 to <=P75 | 179 | 0.50 | 0.72 | 1.03 |
| >P75 | 139 | 0.61 | 0.92 | 1.37 |

The calculated hazard ratios indicate that progression free survival improves in those patients administered bevacizumab in combination with the standard chemotherapy as the tumor specific expression of neuropilin decreases. In Table 4, the upper bound of the 95% confidence interval of treatment hazard ratio in the subset of patients with low tumor specific neuropilin expression (≦median) is below 1. This supports the statistical relevance of the treatment effect (progression free survival) observed in this sub-group of patients.

TABLE 6
Hazard ratios for time to disease progression or death in AVAGAST
patients separated by quartile neuropilin H score (further analysis)

| | | | Hazard | |
|---|---|---|---|---|
| Neuropilin | | Lower | Ratio | Upper Confidence |
| H score | N | Confidence Limit | Estimate | Limit |
| <=P25 | 186 | 0.51 | 0.71 | 0.98 |
| P25 to <=P50 | 164 | 0.47 | 0.68 | 0.98 |
| P50 to <=P75 | 184 | 0.51 | 0.73 | 1.05 |
| >P75 | 145 | 0.60 | 0.89 | 1.33 |

Table 5 was produced in the per-protocol population which excluded patients with major protocol violations. Table 6 was produced in the intent-to-treat population which included all randomized patients. Table 6, therefore, provides a more accurate analysis.

A Kaplan-Meier curve correlating bevacizumab treatment and neuropilin expression with respect to progression free survival is provided in FIG. 2 (median cut-off). The improvement in progression free survival for those patients having relatively low neuropilin expression when bevacizumab is added to the chemotherapy, indicated in the hazard ratios, is also visible in FIG. 2. Median progression free survival was improved by 2.1 months in patients with relatively low tumor specific neuropilin expression (≦median) compared to only 1.3 months for patients with tumor specific neuropilin expression above median. The results demonstrate that the treatment effect (progression free survival) is improved in the subset of patients with relatively low level of neuropilin.

1. A method of selecting a cancer treatment for a patient suffering from gastric cancer, said method comprising:
(a) determining that a sample obtained from the patient has a decreased expression level of neuropilin relative to a control level determined in patients suffering from gastric cancer; and
(b) providing a recommendation that said cancer treatment selected for the patient comprise an effective amount of bevacizumab in combination with a chemotherapy regimen.

2. A method of monitoring patient response to a cancer treatment for a patient suffering from gastric cancer, said method comprising:
(a) determining that a sample obtained from the patient has a decreased expression level of neuropilin relative to a control level determined in patients suffering from gastric cancer; and
(b) providing a recommendation that said cancer treatment for the patient comprise an effective amount of bevacizumab in combination with a chemotherapy regimen.

3. A method for the identification of a patient responsive to, or sensitive to, the addition of bevacizumab treatment to a chemotherapy regimen, said method comprising:
determining an expression level of neuropilin in a patient sample from a patient suspected to suffer from or being prone to suffer from gastric cancer, said patient sample being contacted with EDTA, whereby a decreased level of neuropilin relative to a control level determined in patients suffering from gastric cancer identifies the patient as being responsive to or sensitive to the addition of bevacizumab to said chemotherapy regimen.

4. A method of predicting the response to or sensitivity to the addition of bevacizumab to a chemotherapy regimen of a patient suspected to suffer from, suffering from or prone to suffer from gastric cancer, said method comprising:
determining an expression level of neuropilin in a patient sample from a patient suspected to suffer from or being prone to suffer from gastric cancer, said patient sample being contacted with EDTA, whereby a decreased level of neuropilin relative to a control level determined in patients suffering from gastric cancer predicts the response to or sensitivity to the addition of bevacizumab to said chemotherapy regimen.

5. The method of claim 3 or 4, further comprising informing the patient that they may benefit from cancer treatment comprising an effective amount of bevacizumab in combination with a chemotherapy regimen.

6. The method of any one of claims 1 to 4, wherein said gastric cancer is stomach adenocarcinoma or gastroesophageal junction adenocarcinoma.

7. The method of claim 1, wherein said chemotherapy regimen is a capecitabine-based chemotherapy regimen or a 5-fluorouracil-based chemotherapy regimen.

8. The method of claim 7, wherein said capecitabine-based chemotherapy regimen is a regimen of capecitabine in combination with cisplatin.

9. The method of claim 7, wherein said 5-fluorouracil-based chemotherapy regimen is a regimen of 5-fluorouracil in combination with cisplatin.

10. The method of claim 1, wherein the patient sample is selected from the group consisting of: whole blood, plasma, serum, and combinations thereof.

11. The method of claim 1, wherein said patient sample is selected from the group consisting of gastric tissue resection or gastric tissue biopsy.

12. The method of claim 1, wherein the expression level is a protein expression level.

13. The method of claim 12, wherein the protein expression level is determined by measuring plasma protein level.

14. The method of claim 12, wherein a plasma level of neuropilin in a sample obtained from the patient that is at or below the level of neuropilin in a reference sample, indicates that the patient may benefit from the addition of bevacizumab treatment to said chemotherapy regimen, or has increased likelihood of benefit from the addition of bevacizumab treatment to said chemotherapy regimen.

15. The method of claim 1, wherein said neuropilin expression level is detected by an immunohistochemical method (IHC).

16. The method of claim 1, further comprising administering a therapeutically effective amount of bevacizumab in combination with a chemotherapy to the patient having a decreased level of neuropilin relative to a control level determined in patients suffering from gastric cancer.

17. The method of claim 16, wherein said patient is being co-treated with one or more anti-cancer therapies.

18. The method of claim 17, wherein said anti-cancer therapy is radiation.

19. The method of claim 1, wherein the expression level of neuropilin is determined before neoadjuvant or adjuvant therapy.


SEQUENCE LISTING (1 sequence)

SEQ ID NO: 1 - protein, 923 aa, Homo sapiens
MERGLPLLCAVLALVLAPAGAFRNDKCGDTIKIESPGYLTSPGYPHSYHPSEKCEWLIQAPDPYQRIMINFNPHFDLEDRDCKYDYVEVFDGENENGHFRGKFCGKIAPPPVVSSGPFLFIKFVSDYETHGAGFSIRYEIFKRGPECSQNYTTPSGVIKSPGFPEKYPNSLECTYIVFVPKMSEIILEFESFDLEPDSNPPGGMFCRYDRLEIWDGFPDVGPHIGRYCGQKTPGRIRSSSGILSMVFYTDSAIAKEGFSANYSVLQSSVSEDFKCMEALGMESGEIHSDQITASSQYSTNWSAERSRLNYPENGWTPGEDSYREWIQVDLGLLRFVTAVGTQGAISKETKKKYYVKTYKIDVSSNGEDWITIKEGNKPVLFQGNTNPTDVVVAVFPKPLITRFVRIKPATWETGISMRFEVYGCKITDYPCSGMLGMVSGLISDSQITSSNQGDRNWMPENIRLVTSRSGWALPPAPHSYINEWLQIDLGEEKIVRGIIIQGGKHRENKVFMRKFKIGYSNNGSDWKMIMDDSKRKAKSFEGNNNYDTPELRTFPALSTRFIRIYPERATHGGLGLRMELLGCEVEAPTAGPTTPNGNLVDECDDDQANCHSGTGDDFQLTGGTTVLATEKPTVIDSTIQSEFPTYGFNCEFGWGSHKTFCHWEHDNHVQLKWSVLTSKTGPIQDHTGDGNFIYSQADENQKGKVARLVSPVVYSQNSAHCMTFWYHMSGSHVGTLRVKLRYQKPEEYDQLVWMAIGHQGDHWKEGRVLLHKSLKLYQVIFEGEIGKGNLGGIAVDDISINNHISQEDCAKPADLDKKNPEIKIDETGSTPGYEGEGEGDKNISRKPGNVLKTLDPILITIIAMSALGVLLGAVCGVVLYCACWHNGMSERNLSALENYNFELVDGVKLKKDKLNTQSTYSEA